(19) United States
(12) Patent Application Publication
(10) Pub. No.: US 2019/0099377 A1
(43) Pub. Date: Apr. 4, 2019

(54) METHOD FOR DRY POWDER COATING OSMOTIC DRUG DELIVERY SYSTEM

(71) Applicant: Powder Pharma Coating Inc., Scarborough (CA)

(72) Inventors: JINGXU ZHU, LONDON (CA); YINGLIANG MA, LONDON (CA); QINGLIANG YANG, LONDON (CA); KWOK YUI CHOW, MISSISSAUGA (CA)

(21) Appl. No.: 15/722,526

(22) Filed: Oct. 2, 2017

(51) Int. Cl. A61K 9/48; A61K 9/00; A61K 47/69; A61K 47/32; A61K 47/36

(52) U.S. Cl. CPC A61K 9/4816; A61K 9/0004; A61K 9/4891; A61K 47/36; A61K 47/6943; A61K 47/32; A61K 9/4866

(57) ABSTRACT

The present disclosure provides a process of producing a dry powder coated solid, oral administered pharmaceutical osmotic drug delivery system. The method includes preparing a dry powder film forming polymer coating composition to be coated onto an outer surface of the cores, a size of the coating powder being in a range from about 1 nm to about 500 μm. The osmotic drug delivery cores are placed into an interior of a rotatable housing and the cores may be preheated. The dry powder coating composition is sprayed into the interior of the housing while the housing is rotating to produce a uniform coating of the dry powder coating composition on the outer surface of the cores. The coated cores are cured form a substantially uniform cured film after which one or more orifices are produced through the uniform cured film to expose the outer surface of the cores at a position of each of the one or more orifices.

### FIELD

The present disclosure provides dry powder coating of osmotic drug delivery systems (ODDS).

### BACKGROUND

During the past 50 years, pharmaceutical coating has transitioned from sugar coating to film coatings based on organic solvents (to give organic film coatings) or film coatings based water (to give aqueous film coatings). At present, the process of aqueous film coating has largely replaced organic film coating for pharmaceutical products in consideration of the toxicity, explosion risk, high cost and environmental impact of organic solvents. However, the aqueous film coating process still possesses many limitations such as high energy consumption, high capital equipment cost, risk of microbial contamination and high operating costs. Also the aqueous coating process is not suitable for certain moisture sensitive drugs and a number of products such as osmotic drug delivery systems (ODDS) and many multiparticulate drug delivery systems.

An osmotic drug delivery system (ODDS) is an oral drug delivery system consisting of a solid osmotic core containing drug(s) and osmotic agent(s) surrounded by a continuous coating film with (elementary ODDS) or without a continuous coating film but instead a porous outer film (porosity ODDS) to give a plurality of micropore delivery orifice(s). As the ODDS passes through the gastrointestinal tract (GIT), water is absorbed through the coating film via osmosis, and the resulting osmotic pressure is used to push the active drug through the orifice(s) and/or the coating film. ODDS has gained tremendous attention owing to its distinct characteristics such as zero order drug release kinetics and drug release independent of pH, food and GIT motility.

The first ODDS was proposed by Rose and Nelson in 1955 (Rose and Nelson 1955). It was designed as an osmotic dispenser that was capable of delivering a drug solution at a relatively constant rate. Then Higuchi and Theeuwes (Higuchi 1973, Higuchi and Theeuwes 1974) improved the osmotic dispenser and developed the first practical examples of an elementary osmotic pump tablet (Theeuwes 1975) (Malaterre, Ogorka et al. 2009). This elementary ODDS consists of an osmotic pump core containing a drug and an osmotic agent surrounded by a coating film with one drug delivery orifice, such as that shown in FIG. 1A, which can be drilled by mechanical or laser drilling. Because the drug in this elementary ODDS was released from a single delivery orifice, it could produce high local drug concentration in the GIT causing gastrointestinal irritation.

More particularly, as shown in FIG. 1A, a typical elementary osmotic pump consists of the drug, osmotic agent and semipermeable membrane. Also there is a drug delivery orifice on the semipermeable membrane. Upon the elementary osmotic pump tablets contacting with aqueous media, water immediately diffuses into the core through the semipermeable membrane, dissolving the osmotic agents and generating osmotic pressure, which governs drug release from the delivery orifice at a constant rate.

FIG. 1B shows a schematic diagram of a push-pull osmotic pump tablet. Both embodiments shown in FIGS. 1A and 1B require only one drug delivery orifice as shown. Upon the push-pull osmotic pump tablets contacting with aqueous media, water diffuses into the core through the semipermeable membrane, dissolving the osmotic agents in the lower part of the tablet core and generating osmotic pressure to push the drugs in the upper part of the tablet core to release from drug delivery orifice at the top.

Wang and Jiang (Wang and Jiang 2010) designed a porosity ODDS that allowed drugs to permeate throughout a porous membrane, such as that shown in FIG. 2, that was composed of cellulose acetate and forming using pore-forming agents. As a result, gastro-intestinal (GI) irritation could be minimized or eliminated.

More particularly, as shown in FIG. 2, micropores may be formed in situ immediately after the coated porosity ODDS is exposed to water, through which the drug release could be achieved. The dose dumping can be minimized or eliminated, and also the local drug concentration may be decreased to a safe range due to the uniform distribution of the drug release apertures on the semipermeable membrane.

Cellulose esters are widely used as coat forming materials for ODDS. These polymers have high glass transition temperatures and poor water solubility. It is generally not suitable for use in aqueous coatings. Therefore, solvent coating is considered as the only viable method that can be employed to turn cellulose esters into a coating film on ODDS. Typically, coating polymers and other coating excipients are dissolved in an organic solvent such as acetone to produce a coating solution. Then the solution is sprayed onto the surface of rolling ODDS cores in a pan coater to allow film formation after solvent evaporation. The coated cores are then cured under a controlled temperature and humidity condition in an oven.

Although solvent coating is widely used to produce ODDS with a fairly uniform coating film, it possesses many limitations. The use of organic solvent causes environmental, toxicological and operational safety issues. Also the solvent handling and removal process requires sophisticated containment equipment, high energy consumption associated with heating the large amount of air needed for drying and curing, and complex operational procedures. The cost of building a facility and developing an ODDS product is considered cost prohibitive in the pharmaceutical industry.

It would be advantageous to provide a method of producing osmotic drug delivery systems which is more economical than the wet coating methods in order to avoid use of problematic organic solvents.

### SUMMARY

The present disclosure provides a method of dry powder coating of osmotic drug delivery system (ODDS) products, including ODDS core compositions and the coating, using dry powder coating technology, preferably electrostatic powder coating technology, for oral administration. The purposes of the present disclosure is to provide oral pharmaceutical or nutraceutical products of film coated ODDS.

In an embodiment there is provided a process of producing a dry powder coated solid, oral administered pharmaceutical osmotic drug delivery system, comprising:

preparing a dry powder film forming polymer coating composition to be coated onto an outer surface of the cores, a size of the coating powder being in a range from about 1 nm to about 500 μm;

placing osmotic drug delivery cores into an interior of a rotatable housing and preheating the cores;

electrostatically spraying the dry powder coating composition into the interior;

rotating the housing to produce a uniform coating of the dry powder coating composition on the outer surface of the cores; and

curing the dry coated cores to form a substantially uniform cured film.

The cores may be preheated to a temperature close to a glass transition temperature (T_{g}) of the polymer(s) contained in the film forming polymer coating composition, wherein the polymers are selected to have a glass transition temperature in a range from about 20 to about 200° C.

The glass transition temperature is in a range from about from 30 to about 100° C.

The glass transition temperature is in a range from about from about 40 to about 60° C.

The method may include spraying a suitable amount of plasticizer into the housing to comingle with the dry powder film forming polymer coating composition. The plasticizer may sprayed into the housing prior to spraying the dry powder film forming polymer coating composition, or it may be sprayed into the housing at the same time with spraying the dry powder film forming polymer coating composition.

The plasticizer may be any one or combination of a liquid pure plasticizer, a plasticizer in a solution, and a dry powder plasticizer.

During curing in the housing the coated cores may be cured at a temperature in a range from about 30 to about 100° C., and wherein a curing time is up to about 4 hours.

The one or more orifices are micropores having a diameter in a range from about 1 nm to about 100 μm, or in a range from about 10 nm to about 10 μm, or in a range from about 50 nm to 5 μm.

The one or more orifices have a diameter in a range from about 50 μm to about 2 mm, or in a range from about 100 μm to about 1 mm, or in a range from about 500 μm to 1 mm.

The dry powder film forming polymer coating composition may include pore forming agents.

One or more orifices may be formed through the uniform cured film to expose the outer surface of the cores at a position of each of the one or more orifices. The one or more orifices may be produced by using any one of mechanical drilling, and laser drilling, and an indentation method.

The one or more orifices may be produced by using any one of mechanical drilling, and laser drilling.

A further understanding of the functional and advantageous aspects of the present disclosure can be realized by reference to the following detailed description and drawings.

### BRIEF DESCRIPTION OF THE DRAWINGS

Embodiments disclosed herein will be more fully understood from the following detailed description thereof taken in connection with the accompanying drawings, which form a part of this application, and in which:

FIG. 1a shows a schematic diagram of elementary osmotic pump tablet.

FIG. 1b shows a schematic diagram of a push-pull osmotic pump tablet.

FIG. 2 shows a schematic representation of porosity osmotic pump tablet.

FIG. 3 displays a dissolution profile of salbutamol sulfate from powder coated elementary ODDS of Example 1; (coating polymer: cellulose acetate;

coating level: 3% and 4%).

FIG. 4 displays a dissolution profile of ibuprofen from powder coated elementary ODDS of example 2; (coating polymer: cellulose acetate; coating level: 5.5% and 7.8%).

FIG. 5 displays a dissolution profile of salbutamol sulfate from powder coated porosity ODDS of example 3; (coating polymer: cellulose acetate; coating level: 4%; pore forming agent ratio: 10% and 15%).

FIG. 6 shows a schematic representation of apparatus used for powder coating of ODDS.

### DETAILED DESCRIPTION

Various embodiments and aspects of the disclosure will be described with reference to details discussed below. The following description and drawings are illustrative of the disclosure and are not to be construed as limiting the disclosure. The drawings are not to scale. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions.

As used herein the phrase “osmotic drug delivery system” (ODDS) refers to an oral drug delivery system which includes a solid osmotic core containing drug(s) or active pharmaceutical agent(s) and osmotic agent(s). Typically, an ODDS includes a core and a coating film with drug delivery orifices (one or more) and/or many micropores. The ODDS core of this disclosure comprises at least one active pharmaceutical ingredient (API) and may contain other non-medicinal ingredients, including but not limited to, osmotic agent(s), swellable and non-swellable polymers, solubility enhancers, bioavailability enhancers, diluents, binders and lubricants. These components are mixed directly and/or granulated (wet or dry using a granulator or roller compactor), and compressed using a tableting machine.

A typical size regime of these ODDS dosage forms are in a range from about 100 nm to about 50 mm, preferably from 1 mm to 20 mm, more preferably from 3 mm to 10 mm.

The phrase “active agont” and “active ingredient” refer to active pharmaceutical ingredients (APIs) or drugs.

The phrase “film forming coating powder” and “polymer powder” refers to the powder being used to form the coating film on the ODDS and can optionally contain other constituents or materials including but not limited to talc powder, pigments, flowability additives and dry powder plasticizers.

The phrase “pore forming agent” refers to the powdered polymers, or liquid polymers, or polymer solutions with small molecular weight that can be used as the pore forming agent in the pharmaceutical coating process. Pore forming agents are water soluble materials which can be sprayed together with coating powders including film forming materials in the powder coating process. After being cured, they would be part of the coating film. After being swallowed and upon contacting with GI tract, those pore forming agents are dissolved and leached out, leaving lots of small holes (micropores) on the film, hence the coating film becomes permeable allowing fluids to move into and dissolve the core thereby releasing the active drug or agent.

The phrase “drug delivery orifice” refers to an orifice with a typical diameter of between about 200 μm to about 1 mm located on the coating film which can be created by many techniques, including but not limited to, mechanical drilling or a laser drilling or indentation method or any other methods.

The phrase “micropores” refers to the pores located on the coating film formed by the pore forming agent during coating process, ranged from 1 nm to 100 μm, preferably from 10 nm to 10 μm, more preferably from 50 nm to 5 μm.

The term “curing” means applying an energy source, generally a heat source but it may also be an ultraviolet source or any other source, to increase the temperature of the coated solid dosage forms, so as to solidify or partially solidify the coating on the surface of the dosage. Such heat source, for instance, can be a heating element inside the chamber of the rotatable housing where the coatings are applied, or outside the housing but close enough to be able to transfer heat to the dosage forms inside the housing, or a hot air flowing through the chamber. For polymer powders sensitive to ultraviolet waves, an ultraviolet source may also be used as an energy source for curing.

Eudragit® is a trade mark of Evonik.

With a goal of providing an alternative to form the coating film for osmotic drug delivery system (ODDS) using a powder coating technique, a preferred coating technique being an electrostatic powder coating technique so that organic solvents and water can be avoided based methods of coating are not required.

After the preparation of ODDS core a powder coating process us used to obtain the out layer coating of the ODDS wherein the coating material contains one or more of film formation polymers, flavoring agents, taste modifying agents, taste masking agents, pH sensitive coating materials, moisture barrier coating materials or a combination thereof.

The powder coating process, particularly electrostatic powder coating process comprises the following steps.

A) Preparation of the powdered coating material is the first step, and in an embodiment the coating powder may be milled using a suitable mill such as an airjet mill, grinder ball mill, pin mill, hammering mill or combination thereof to give particles in a preselected size range. The particle size of coating powder can be in a range of about 1 nm to about 200 μm, preferably in a range of about 10 to about 100 μm, and more preferably in a range from about 20 to about 40 μm. After particle size reduction, those coating materials are mixed together to form a coating formulation.

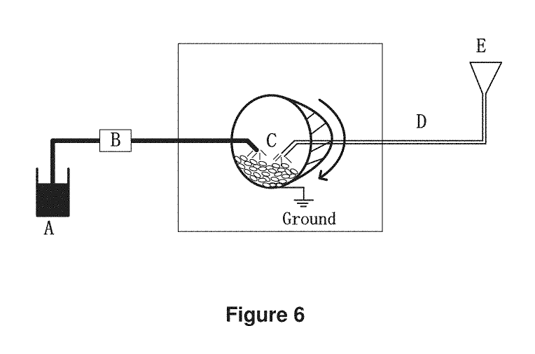

B) Positioning and preheating is accomplished by loading the ODDS cores into a rotatable housing (for example, a coating pan shown in FIG. 6) which has been preheated to a temperature close to the glass transition temperature (T₉) of the coating polymers, which is typically in a range from about 30 to about 100° C., preferably from about 30 to about 80° C., more preferably from about 40 to about 60° C.

C) During coating powder deposition the adhesion of the coating powders may need the assistance of a suitable amount of dry powdered plasticizer, or liquid plasticizer or plasticizer solution with a weight ratio range of 0% to about 200% based on weight of the film forming coating powders, preferably in a range from about 5% to about 100%, more preferably in a range from about 10% to about 80%, and in particular preferably in a range of about 20% to about 60%. Plasticizer(s), when present, and film forming coating powders are sprayed onto the surface of the ODDS cores using an air atomizing or airless spray nozzle/electrostatic spray gun (e.g. corona charging gun or a tribo charging gun). If corona gun is used, the voltage can be in a range of about 20 to about 120 kilo Volts (kV), preferably in a range of about 25 to about 70 kV, more preferably in a range of about 40 to about 70 kV, and in particular preferably in a range of about 50 to about 70 kV. The plasticizer and coating powders may be sprayed either simultaneously, or via the alternating spray method wherein the plasticizer or powered polymer material is sprayed first and then the other is sprayed and the process may be repeated. Alternatively, plasticizer can be mixed with powdered material and then this mixture can be sprayed onto the ODDS. In all cases, heating preferably continues during the spraying of plasticizer and powdered materials.

D) After the deposition of coating powders, ODDS remains in the rotatable housing under a curing temperature, which is in a range from about 30 to about 100° C., preferably from 30 to 80° C., more preferably from about 40 to about 60° C., for a period of time ranged from 0 to about 10 hours, preferably from about 0 to about 4 hours, more preferably from about 1 to about 2 hours, to allow those deposited coating powders to coalesce and form the coating film.

E) Pore formation may be accomplished several ways. After the film formation step, if a pore forming agent was not included in the film forming coating powder formulation, the coated ODDS can be drilled in the center using a mechanical drill or a laser drill or indentation method (a recess or notch on the surface of the semipermeable membrane). to achieve the drug deliver orifice, ranged from 50 μm to 2 mm, preferably from 100 μm to 1 mm, more preferably from 500 μm to 1 mm.

The ODDS core will contain at least one active agent. Typical pharmaceutically active agents include, but are not limited to, e.g., anti-inflammatory, antipyretic, anticonvulsant and/or analgesic agents such as indomethacin, diclofenac, diclofenac Na, ibuprofen, and anti-asthma drugs such as salbutamol and so on. Other APIs having the same or different physiological activity as those above, or suitable mixture thereof, can also be employed in this invention. As used herein, the term “active agent” includes all pharmaceutically acceptable forms of the active agent being described. For example, the active agent can be in an isomeric mixture, a solid complex bound to an ion exchange resin, or the like. In addition, the active agent can be in a solvated form.

The active agent can be in any suitable form. For example, it can be in the form of a powder, pellet, or a granule (i.e., an aggregate of smaller units of active agent), or small tablets or any combination of any thereof. These osmotic drug delivery systems cores also contain one or more osmotic agents. The motive force of the ODDS of the present disclosure depends on the osmotic pressure generated by the solution of the osmotically effective solute contained in the coating film, which solution exhibits an osmotic pressure gradient against water. To maintain the solution saturated and therefore to achieve a constant osmotic pressure throughout operation of the ODDS, the membrane or bag containing the solution also contains excess solute in solid form. The osmotically effective solute can include, but not limit to, sodium chloride, potassium chloride, lithium chloride, magnesium chloride, magnesium sulfate, calcium bicarbonate, sodium sulfate, calcium sulfate, and mixtures thereof. The excess solid solute can be in the form of dispersed particles or preferably in the form of a pellet. The solution can initially be a solution of the same or of an osmotically effective solute different than the solid excess solute.

The ODDS core may also include one or more functional excipients such as compressible agent, lubricants, thermal lubricants, antioxidants, binders, diluents, sweeteners, chelating agents, colorants, flavorants, surfactants, solubilizers, wetting agents, stabilizers, hydrophilic polymers, hydrophobic polymers, waxes, lipophilic materials, absorption enhancers, protease inhibitors, preservatives, absorbents, cross-linking agents, bioadhesive polymers, retardants, and fragrance.

The composition of the coating powders may include coating powders which include anti-tacky agents, and other additives such as pigments, plasticizers, pore forming agents, coating powder glidants or any combinations of any thereof. The coating polymers may be chosen that provide flavoring or taste modifying/masking or moisture barrier include, but not limited to, methylcellulose, hydroxyethyl cellulose, hydroxypropyl cellulose (HPC), hydroxylpropyl methyl cellulose (HPMC) and so on to give a few non-limiting examples.

Coating polymers that can be used to form the coating film of the ODDS may include cellulose esters such as cellulose acetate, ethylcellulose and cellulose derivatives such as cellulose nitrate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, and Eudragit® RL, Eudragit® RS or any combination of any thereof.

Exemplary pore forming agents include water soluble polymers such as methylcellulose, hydroxyethyl cellulose, hydroxypropyl cellulose (HPC), hydroxylpropyl methyl cellulose (HPMC), poly(vinylpyrrolidinone) (PVP), polyethylene glycols such as but not limited to PVP, PEG 400, PEG 600, PEG 3350, propylene glycol, polaxamer and povidone; binders such as lactose, calcium sulfate, calcium phosphate and the like; salts such as sodium chloride, magnesium chloride and the like to give a few examples, and any combinations thereof and other similar or equivalent materials which are widely known in the art.

The plasticizer is used to reduce the glass transition temperature of the coating polymer. Plasticizer can be solid, liquid or plasticizer solution. When the plasticizer is liquid polymers or polymer solutions, it can also be used to decrease the electrical resistivity of the ODDS so that the adhesion of coating powder and the coating efficiency could be promoted. Furthermore, liquid plasticizers or plasticizer solutions can provide a strong capillary force between particles and surface of the ODDS, enhancing coating powder adhesion. Plasticizers suitable for use in the present invention include, but are not limited to glycerol, propylene glycol, PEG 200-600 grades, triacetin, diethyl phthalate (DEP), dibutyl phthalate (DBP) and tributyl citrate (TBC), triethyl citrate (TEC) and so on.

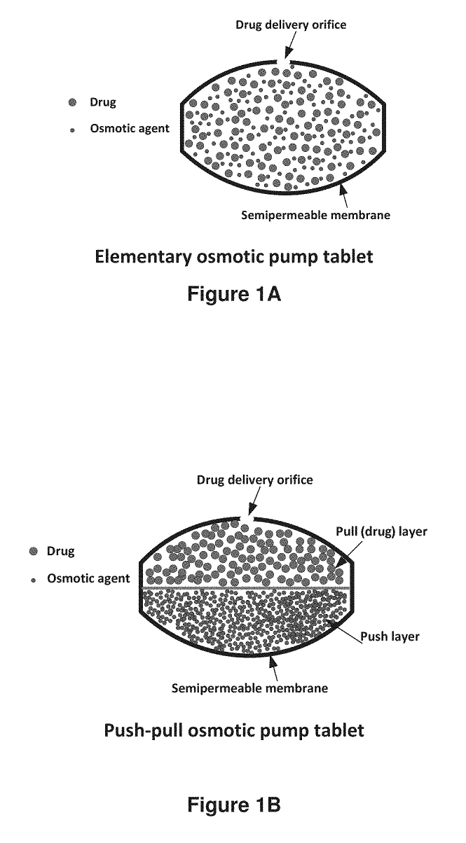

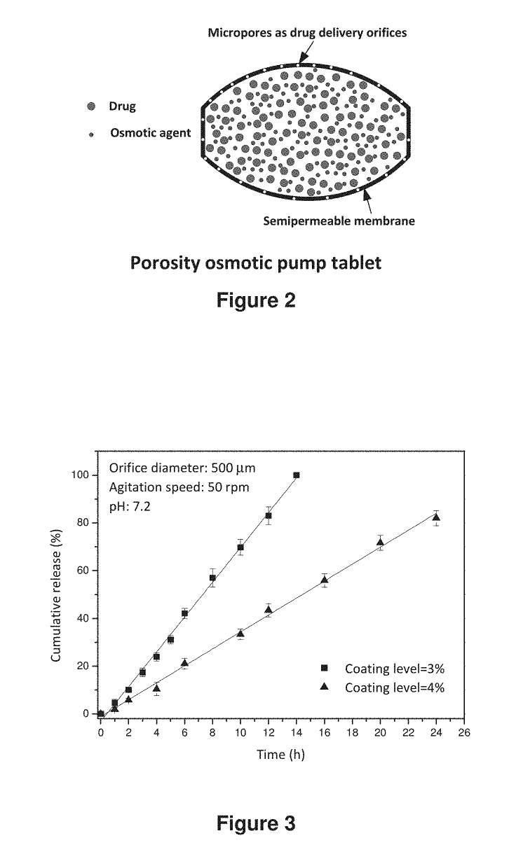

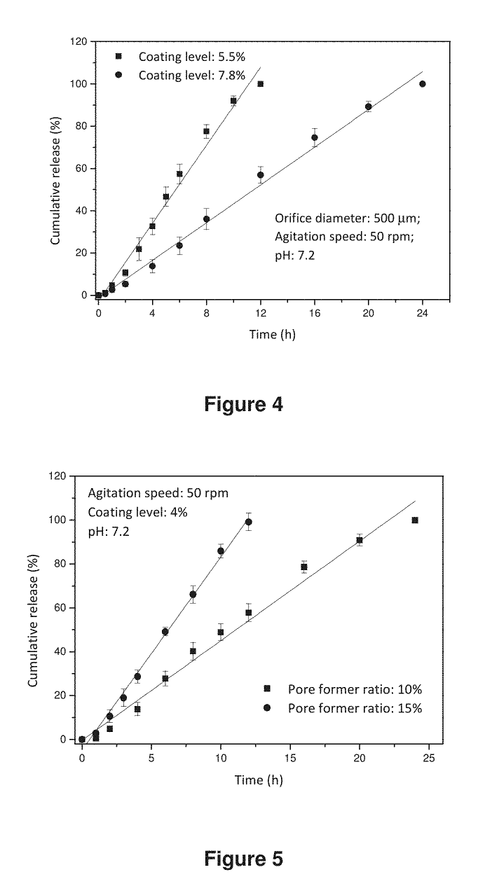

For elementary osmotic pump and push-pull osmotic pump tablets as shown in FIGS. 1A and 1B respectively, there will be a drug delivery orifice on the semipermeable membrane. While for porosity osmotic pump as shown in FIG. 2, there is no need for a well defined drug delivery orifice through the membrane, but the membrane (the coating film) is produced to be porous with many micropores present.

It will be understood that compounds used in the art of pharmaceutical formulations generally serve a variety of functions or purposes. Thus, if a compound named herein is mentioned only once or is used to define more than one term herein, its purpose or function should not be construed as being limited solely to that named purpose(s) or function(s).

The present process will now be illustrating using the following non-limiting examples.

### Example 1

### Preparation of the ODDS Core

Salbutamol sulfate, sodium chloride, microcrystalline cellulose and Plasdone K-29/32 (PVP) were mixed together and passed through a 30-mesh sieve. Then the mixture was granulated using a granulator (Mechanomill MM-20N, OKADA SEIKO Co. Ltd, Iwate, Japan) with a speed of 300 rpm for 6 minutes, followed by adding magnesium stearate and mixing for another 3 min with same speed. The ready formulation was then compressed into tablets (diameter: 5 mm; weight: 180±5 mg) by using a tablet machine with one punch (Shanghai Tianxiang & Chentai Pharmaceutical Machinery Co. Ltd, Shanghai, China).

TABLE 1
Composition of ODDS used Example 1 & 3

| Ingredient | Function | % w/w |
|---|---|---|
| Salbutamol sulfate | Active | 20 |
| | ingredient | |
| Sodium chloride | Osmotic agent | 30 |
| Avicel ® Microcrystalline | Diluent | 43 |
| cellulose PH-102 | | |
| Plasdone K-29/32 (PVP) | Binder | 6.5 |
| Magnesium stearate | Lubricant | 0.5 |

### Powder Coating Process

Before the coating process, the particle size of coating material (cellulose acetate, Eastman™ CA 398-10) was reduced by a blade grind mill, prior to use. Particle size of the coating materials was confirmed by a particle size analyzer (TSI Corporation, Model 3603, Shoreview, Minn., USA). The particle size at 50% of total weight fraction was used as average particle size (the median value). The average diameter of cellulose acetate and talc was 26.7 and 28.9 μm, respectively. After particle size reduction, cellulose acetate was mixed together with talc powder, colloidal silicon dioxide (AEROSIL® 200 Pharma) and pigment to form a coating powder. Before loading of coating powder, the osmotic pump tablet cores (60 g) were firstly loaded into a coating pan (shown as FIG. 6) and preheated to a temperature of 60° C. After preheating, liquid plasticizer (triethyl citrate with a flowrate of 0.5 g/min for 2 min) was sprayed onto the surface of the tablets, immediately followed by the spraying of coating powder (2 g). Then the tablets were further cured for 1-2 hours at 60° C. to allow the deposited particles coalesce together to form a uniform film.

TABLE 2
Formulation of the coating powders for the Example 1 & 2

| Ingredient | % w/w |
|---|---|
| Cellulose acetate (Eastman ™ CA 398-10) | 80% |
| Talc powder | 19% |
| Pigment (FD&C Blue # 1) | 0.5% |
| Colloidal silicon dioxide (AEROSIL ® 200 | 0.5% |
| Pharma) | |

### Creation of the Drug Delivery Orifice

The dry powder coated osmotic pump tablets were drilled in the center to form a drug delivery orifice using a mechanical drill (500 μm).

### Example 2

Both the preparation of the tablet core process, powder coating process and the creation of the drug delivery orifice are the same as Example 1.

TABLE 3
Composition of ODDS used Example 2

| Ingredient | Function | % w/w |
|---|---|---|
| Ibuprofen | Active | 20 |
| | ingredient | |
| Sodium chloride | Osmotic agent | 30 |
| Avicel ® Microcrystalline | Diluent | 43 |
| cellulose PH-102 | | |
| Plasdone K-29/32 (PVP) | Binder | 6.5 |
| Magnesium stearate | Lubricant | 0.5 |

### Example 3

The preparation of the tablet core process is the same as Example 1. While for the coating process, PEG 3350 (10% & 15%) was added as the pores former in the coating powder. The coating process is the same as Example 1 except the coating powder formulation and there is no need to create the drug delivery orifice. The coating level is 4%.

TABLE 4
Formulation of coating powders for Example 3

| | Ingredient (% w/w) | | | | |
|---|---|---|---|---|---|
| | Cellulose | | | | Colloidal silicon |
| | acetate | | | Pigment | dioxide |
| | (Eastman ™ | PEG | | (FD&C | (AEROSIL ® |
| Formulation | CA 398-10) | 3350 | Talc | Blue # 1) | 200 Pharma) |
| 1 | 80% | 10% | 9% | 0.5% | 0.5% |
| 2 | 75% | 15% | | | |

### Dissolution Tests

For those four examples, the release profile of drug from osmotic pump tablets after coated with various coating conditions was determined using the USP<711> Method A (paddle method; rotation of 50 rpm; pH=7.2 phosphate buffer solution; 37° C., n=6). At predetermined time interval, samples were withdrawn (10 ml, replaced) and assayed spectrophotometrically at 276 nm (UV-vis, Aliegment, USA).

The present method of producing dry powder coated osmotic pump tablets is advantageous for many reasons, for example there are significant energy savings without having to use any fluidizing hot air, it is an environmental, friendly as there is no need to use any organic solvents. The present process is very economical as powder coated products can be produced cheaply compared to those in the market prepared using current technology.

The process is a simple and controllable coating process, which gives low air handling equipment cost without using any associated pre- and post-air treatment equipment. The present process may be easily retrofitted into a typical pharmaceutical liquid coating facility to provide a fast track to commercialization. The resulting coatings are very uniform and provide a better appearance of the end product using the ultrafine coating powders. A further advantage of the dry powder coating method of coating the osmotic delivery system observed by the inventors is that the final coating film more consolidated so that films typically thinner than those produced by other wet techniques may be produced. This dry method is very advantageous for producing coated ODDS's which have very moisture sensitive active pharmaceutical ingredients and/or excipients.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCES

Higuchi, T. (1973). Osmotic dispenser with collapsible supply container. U.S. Pat. Nos. 3,760,805 3,760,805 A.
Higuchi, T. and F. Theeuwes (1974). Osmatic dispensing device for releasing beneficial agent. U.S. Pat. No. 3,845,770. U.S. Pat. No. 3,845,770 A.
Malaterre, V., et al. (2009). “Oral osmotically driven systems: 30 years of development and clinical use.” European Journal of Pharmaceutics and Biopharmaceutics 73(3): 311-323.
Rose, S. and J. F. Nelson (1955). “A Continuous Long-Term Injector.” Austral. J. Biol. 33: 415-420.
Theeuwes, F. (1975). “Elementary Osmotic Pump.” Journal of Pharmaceutical Sciences 64(12): 1987-1991.
Wang, J. and H. Jiang (2010). Controlled porous osmotic pump tablets of high permeable drugs and the preparation process thereof. U.S. Pat. No. 8,703,193 B2.
Zhu, J., et al. (2012). Direct coating solid dosage forms using powdered materials. U.S. Pat. No. 8,161,904.

Therefore what is claimed is:

1. A process of producing a dry powder coated solid, oral administered pharmaceutical osmotic drug delivery system, comprising:
preparing a dry powder film forming polymer coating composition to be coated onto an outer surface of the cores, a size of the coating powder being in a range from about 1 nm to about 500 μm;
placing osmotic drug delivery cores into an interior of a rotatable housing and preheating the cores;
spraying the dry powder coating composition into the interior;
rotating the housing to produce a uniform coating of the dry powder coating composition on the outer surface of the cores; and
curing the dry coated cores to form a substantially uniform cured film.

2. The method according to claim 1 wherein the cores are preheated to a temperature close to a glass transition temperature (T_{g}) of the polymer(s) contained in said film forming polymer coating composition, wherein said polymers are selected to have a glass transition temperature in a range from about 20 to about 200° C.

3. The method according to claim 2 wherein said glass transition temperature is in a range from about from 30 to about 100° C.

4. The method according to claim 2 wherein said glass transition temperature is in a range from about from about 40 to about 60° C.

5. The method according to claim 1, including spraying a suitable amount of plasticizer into the housing to comingle with the dry powder film forming polymer coating composition.

6. The method according to claim 5, wherein said plasticizer is sprayed into the housing prior to spraying the dry powder film forming polymer coating composition.

7. The method according to claim 5, wherein said plasticizer is sprayed into the housing at the same time with spraying the dry powder film forming polymer coating composition.

8. The method according to claim 1, including spraying a suitable amount of plasticizer into said housing, said suitable amount of plasticizer being selected to reduce a glass transition temperature (T_{g}) of the dry powder film forming polymer coating composition to a range between about 30 to about 100° C.

9. The method according to claim 8, wherein said plasticizer is any one or combination of a liquid pure plasticizer, a plasticizer in a solution, and a dry powder plasticizer.

10. The method according to claim 1, wherein during curing in the housing the coated cores are cured at a temperature in a range from about 30 to about 100° C., and wherein a curing time is up to about 4 hours.

11. The method according to claim 10, wherein during curing in the housing the coated cores are cured at a temperature in a range from about 40 to about 60° C.

12. The method according to claim 1, wherein the one or more orifices are micropores having a diameter in a range from about 1 nm to about 100 μm.

13. The method according to claim 12, wherein the micropores have a diameter in a range from about 10 nm to about 10 μm.

14. The method according to any one of claim 12 wherein the micropores have a diameter in a range from about 50 nm to 5 μm.

15. The method according to claim 1, wherein the one or more orifices have a diameter in a range from about 50 μm to about 2 mm.

16. The method according to claim 1, wherein the one or more orifices have a diameter in a range from about 100 μm to about 1 mm.

17. The method according to claim 1, wherein the one or more orifices have a diameter in a range from about 500 μm to 1 mm.

18. The method according to claim 1, wherein the dry powder film forming polymer coating composition comprises pore forming agents.

19. The method according to claim 1, including producing one or more orifices through the uniform cured film to expose the outer surface of the cores at a position of each of the one or more orifices, and wherein the one or more orifices are produced by using any one of mechanical drilling, and laser drilling, and an indentation method.

20. The method according to claim 1, wherein the spraying the dry powder film forming polymer coating composition is accomplished by electrostatically spraying.

21. The method according to claim 20 wherein the electrostatically spraying is performed using an electrostatic spray gun.

22. The method according to claim 21, wherein the electrostatic spray gun is a corona charging gun or a tribo charging gun.

23. The method according to claim 1, wherein the pore forming agents include any one or combination of
water soluble polymers comprising methylcellulose, hydroxyethyl cellulose, hydroxypropyl cellulose (HPC), hydroxylpropyl methyl cellulose (HPMC), and poly(vinylpyrrolidinone) (PVP),
polyethylene glycols such as but not limited to PVP, PEG 400, PEG 600, PEG 3350, propylene glycol, polaxamer and povidone;
binders such as lactose, calcium sulfate, calcium phosphate and the like; salts such as sodium chloride, magnesium chloride and the like to give a few examples.

24. The method according to claim 1, wherein the film forming polymers include
cellulose acetate, ethylcellulose and cellulose derivatives such as cellulose nitrate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, Eudragit® RL, Eudragit® RS or any combination of any thereof.

25. An oral administered pharmaceutical osmotic drug delivery system to be powder coated using the method of claim 1.

26. A dry powder film forming polymer coating composition to be powder coated onto an outer surface of the cores, comprising:
cellulose esters, Eudragit® RL, Eudragit® RS, pore forming agents, colloidal silicon dioxide and pigments, or any combination of any thereof.
